(19) United States
(12) Patent Application Publication
(10) Pub. No.: US 2010/0137779 A1
(43) Pub. Date: Jun. 3, 2010

(54) SYSTEMS, DEVICES, AND METHODS FOR POWERING AND/OR CONTROLLING DEVICES, FOR INSTANCE TRANSDERMAL DELIVERY DEVICES

(76) Inventor: Forrest Seitz, Beaverton, OR (US)

Correspondence Address: SEED INTELLECTUAL PROPERTY LAW GROUP PLLC, 701 FIFTH AVE, SUITE 5400, SEATTLE, WA, 98104, US

(21) Appl. No.: 12/556,374

(22) Filed: Sep. 9, 2009

Related U.S. Application Data
(60) Provisional application No. 61/095,526, filed on Sep. 9, 2008.

(51) Int. Cl. A61N 1/30; H02K 33/02; A61N 1/04

(52) U.S. Cl. 604/20; 335/229; 607/115

(57) ABSTRACT

Systems, devices, and methods for powering and/or controlling active or electrically powered transdermal delivery devices employ a magnetic field blocker such as a ferrous disk between a magnetic coupler element and a battery to counter adverse affects on the battery by the magnetic coupler element.

### CROSS REFERENCE TO RELATED APPLICATIONS

This application claims benefit under 35 U.S.C. 119(e) of U.S. Provisional Patent Application Ser. No. 61/095,526, filed Sep. 9, 2008 and entitled “Systems, Devices, and Methods for Powering and/or Controlling Devices, for Instance Transdermal Delivery Devices,” which is incorporated herein by reference in its entirety.

### BACKGROUND

1. Field

This disclosure generally relates to powering and/or controlling devices, for example medical devices, for instance transdermal delivery devices.

2. Description of the Related Art

Medical devices that employ electromotive forces are well known in the art. For example, iontophoretic drug delivery devices employ an electromotive force and/or current to transfer an active agent (e.g., a charged substance, an ionized compound, an ionic drug, a therapeutic, a bioactive-agent, and the like), to a biological interface (e.g., skin, mucus membrane, and the like), by using a small electrical charge applied to an iontophoretic chamber containing a similarly charged active agent and/or its vehicle.

Iontophoresis devices typically include an active electrode assembly and a counter electrode assembly, each coupled to opposite poles or terminals of a power source, for example a chemical battery or an external power station connected to the iontophoresis device via electrical leads. Each electrode assembly typically includes a respective electrode element to apply an electromotive force and/or current. Such electrode elements often comprise a sacrificial element or compound, for example silver or silver chloride. The active agent may be either cationic or anionic, and the power source may be configured to apply the appropriate voltage polarity based on the polarity of the active agent.

Iontophoresis may be advantageously used to enhance or control the delivery rate of the active agent. The active agent may be stored in a reservoir such as a cavity. Alternatively, the active agent may be stored in a reservoir such as a porous structure or a gel. An ion exchange membrane may be positioned to serve as a polarity selective barrier between the active agent reservoir and the biological interface. The membrane, typically only permeable with respect to one particular type of ion (e.g., a charged active agent), prevents the back flux of oppositely charged ions from the skin or mucous membrane.

Commercial acceptance of iontophoresis devices is dependent on a variety of factors, such as cost to manufacture, shelf life, stability during storage, efficiency and/or timeliness of active agent delivery, biological capability, and/or disposal issues. Commercial acceptance of iontophoresis devices is also dependent on their versatility and ease-of-use. Therefore, it may be desirable to have novel approaches for powering iontophoresis devices.

The present disclosure is directed to overcoming one or more of the shortcomings set forth above, and providing further related advantages.

### BRIEF SUMMARY

At least one embodiment may be summarized as a medical device including a first portion including at least one electrode selectively operable to provide an electrical potential and including at least a first element of a magnetic coupler; a second portion including at least one battery cell and including at least a second element of the magnetic coupler, the second element of the magnetic coupler complementary to the first element of the magnetic coupler, the second portion of the medical device selectively removably magnetically coupleable to the first portion of the medical device via a magnetic interaction between the first and the second elements of the magnetic coupler; and a ferrous element physically carried by one of the first or the second portions, wherein at least one of the first or the second elements is a magnet and the ferrous element is physically positioned between the battery and the magnet to steer a magnetic flux of the magnet away from the battery. The second element of the magnetic coupler may be the magnet and the first element of the magnetic coupler may be a ferrous metal. The second element of the magnetic coupler may be the magnet and the first element of the magnetic coupler may be another magnet, where opposite magnet poles of the two magnets face one another when the second portion of the medical device is magnetically coupled to the first portion of the medical device. The first element of the magnetic coupler may be the magnet and the second element of the magnetic coupler may be one of a ferrous metal or another magnet.

The second portion may further include a control circuit configured to control the electrical potential provided at the at least one electrode of the first portion.

The first portion may further include an active agent reservoir positioned such that the electrical potential provided at the at least one electrode of the first portion drives an active agent from the active agent reservoir. The battery may be disc shaped and the ferrous element may be disc shaped. A diameter of the ferrous element may be at least approximately equal to a diameter of the battery. The magnet may be disc shaped and the diameter of the ferrous element may be greater than a diameter of the magnet. At least one of the first or the second portions of the medical device may include a non-ferrous spacer, the non-ferrous spacer positioned between the ferrous element and the battery. The magnet may take the form of at least one permanent magnet. The at least one permanent magnet may be at least one of a high-energy flexible magnet, a neodymium magnet, a ceramic magnet, a samarium cobalt magnet, or an alnico magnet.

The first portion may further include a first and a second electrical contact, and wherein the second portion may include a complementary first and a complementary second electrical contact, the first and the second electrical contacts positioned with respect to the first element of the magnetic coupler and the complementary first and the complementary second electrical contacts positioned with respect to the second element of the magnetic coupler such that the first complementary electrical contact is in electrically conductive communication with the first electrical contact and the second complementary electrical contact is in electrically conductive communication with the second electrical contact when the second portion of the medical device is magnetically coupled to the first portion of the medical device.

At least one embodiment may be summarized as an apparatus including a battery holder configured to hold a battery; a first coupling magnet physically coupled to the battery holder and positioned proximate the battery holder; and a ferrous element positioned between the battery holder and the first coupling magnet to steer a magnetic flux of the first coupling magnet away from the battery holder.

The apparatus may further include a non-ferrous spacer positioned between the ferrous element and the battery holder.

The apparatus may further include a control circuit coupled to control at least one of a current or a voltage delivered from the battery;

The apparatus may further include the battery, received in the battery holder. The apparatus may be a removable power source magnetically coupleable to another device via the first coupling magnet.

The apparatus may be a medical device and may further include at least one electrode operable to provide an electrical potential when coupled to the battery.

The medical device may be a transdermal active agent delivery device and may further include at least one active agent reservoir that stores an active agent. The at least one active agent reservoir that stores an active agent may be carried by a first portion that may be removable attachable to a second portion which carries the battery holder.

The apparatus may further include at least two electrical contacts accessible from an exterior of the apparatus, the electrical contacts in electrical communication with the battery when the battery is held by the battery holder.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements, as drawn, are not intended to convey any information regarding the actual shape of the particular elements, and have been solely selected for ease of recognition in the drawings.

FIG. 1 is an isometric view of an electrically powered or active transdermal deliver device, including an active agent delivery component and a separate portable power supply system, according to one illustrated embodiment.

FIG. 2 is a cross-sectional view of the electrically powered or active transdermal deliver device of FIG. 1 where the portable power supply system is spaced from the active agent delivery component.

FIG. 3 is an exploded view of a portable power supply system according to one illustrated embodiment.

FIG. 4 is a graph showing battery discharge performance for three prototype portable power supply systems.

FIG. 5 is a graph showing magnetic flux steering for various thicknesses of magnetic flux blocking elements and spacers.

FIG. 6 is a functional block diagram of an electrically powered or active transdermal deliver device according to one illustrative embodiment.

FIG. 7 is an electrical schematic diagram of a circuit for a portable power supply system according to one illustrative embodiment.

FIG. 8 is an electrical schematic diagram of a circuit for a portable power supply system according to one illustrative embodiment.

FIG. 9 is a schematic diagram of the transdermal delivery device comprising an active electrode assembly and a counter electrode assembly according to one illustrated embodiment.

### DETAILED DESCRIPTION

In the following description, certain specific details are included to provide a thorough understanding of various disclosed embodiments. One skilled in the relevant art, however, will recognize that embodiments may be practiced without one or more of these specific details, or with other methods, components, materials, etc. In other instances, well-known structures associated with electrically powered transdermal delivery devices including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment,” or “an embodiment,” or “in another embodiment” means that a particular referent feature, structure, or characteristic described in connection with the embodiment is included in at least one embodiment. Thus, the appearance of the phrases “in one embodiment,” or “in an embodiment,” or “in another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to an electrically powered device including “a power source” includes a single power source, or two or more power sources. It should also be noted that the term “or” is generally employed in its sense including “and/or” unless the content clearly dictates otherwise.

As used herein the term “membrane” means a boundary, layer, barrier, or material, which may or may not be permeable. The term “membrane” may further refer to an interface. Unless specified otherwise, membranes may take the form of a solid, a liquid, or a gel, and may or may not have a distinct lattice, non-cross-linked structure, or cross-linked structure.

As used herein the term “ion selective membrane” means a membrane that is substantially selective to ions, passing certain ions while blocking passage of other ions. An ion selective membrane, for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH, and ACS, also from Tokuyama Co., Ltd.

As used herein and in the claims, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two-film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules at a first rate, and some other molecules at a second rate different from the first. In yet further embodiments, the “semi-permeable membrane” may take the form of a selectively permeable membrane allowing only certain selective molecules to pass through it.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three-dimensional network, a colloidal suspension of a liquid in a solid, a semi-solid, a cross-linked gel, a non-cross-linked gel, a jelly-like state, and the like. In some embodiments, the gel matrix may result from a three-dimensional network of entangled macromolecules (e.g., cylindrical micelles). In some embodiments, a gel matrix may include hydrogels, organogels, and the like. Hydrogels refer to three-dimensional network of, for example, cross-linked hydrophilic polymers in the form of a gel and substantially composed of water. Hydrogels may have a net positive or negative charge, or may be neutral.

As used herein and in the claims, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

As used herein and in the claims, the term “active agent” refers to a compound, molecule, or treatment that elicits a biological response from any host, animal, vertebrate, or invertebrate, including, for example, fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., a cosmetic substance, and the like), a vaccine, an immunological agent, a local or general anesthetic or painkiller, an antigen or a protein or peptide such as insulin, a chemotherapy agent, and an anti-tumor agent.

In some embodiments, the term “active agent” refers to the active agent as well as to its pharmacologically active salts, pharmaceutically acceptable salts, prodrugs, metabolites, analogs, and the like. In some further embodiments, the active agent includes at least one ionic, cationic, ionizeable, and/or neutral therapeutic drug, and/or pharmaceutically acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is exhibiting a polarity at one portion relative to another portion. For instance, an active agent having an amino group can typically take the form an ammonium salt in solid state and dissociates into a free ammonium ion (NH4⁺) in an aqueous medium of appropriate pH.

The term “active agent” may also refer to electrically neutral agents, molecules, or compounds capable of being delivered via electro-osmotic flow. The electrically neutral agents are typically carried by the flow of, for example, a solvent during electrophoresis. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include lidocaine, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of active agents include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (NOVOCAINE®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

As used herein and in the claims, the term “subject” generally refers to any host, animal, vertebrate, or invertebrate, and includes fish, mammals, amphibians, reptiles, birds, and particularly humans.

As used herein and in the claims, the term “agonist” refers to a compound that can combine with a receptor (e.g., an opioid receptor, toll-like receptor, and the like) to produce a cellular response. An agonist may be a ligand that directly binds to the receptor. Alternatively, an agonist may combine with a receptor indirectly by forming a complex with another molecule that directly binds the receptor, or otherwise results in the modification of a compound so that it directly binds to the receptor.

As used herein and in the claims, the term “antagonist” refers to a compound that can combine with a receptor (e.g., an opioid receptor, a toll-like receptor, and the like) to inhibit a cellular response. An antagonist may be a ligand that directly binds to the receptor. Alternatively, an antagonist may combine with a receptor indirectly by forming a complex with another molecule that directly binds to the receptor, or otherwise results in the modification of a compound so that it directly binds to the receptor.

As used herein and in the claims, the term “effective amount” or “therapeutically effective amount” includes an amount effective at dosages and for periods of time necessary, to achieve the desired result. The effective amount of a composition containing a pharmaceutical agent may vary according to factors such as the disease state, age, gender, and weight of the subject.

As used herein and in the claims, the term “analgesic” refers to an agent that lessens, alleviates, reduces, relieves, or extinguishes a neural sensation in an area of a subject's body. In some embodiments, the neural sensation relates to pain, in other aspects the neural sensation relates to discomfort, itching, burning, irritation, tingling, “crawling,” tension, temperature fluctuations (such as fever), inflammation, aching, or other neural sensations.

As used herein and in the claims, the term “anesthetic” refers to an agent that produces a reversible loss of sensation in an area of a subject's body. In some embodiments, the anesthetic is considered to be a “local anesthetic” in that it produces a loss of sensation only in one particular area of a subject's body.

As one skilled in the relevant art would recognize, some agents may act as both an analgesic and an anesthetic, depending on the circumstances and other variables including but not limited to dosage, method of delivery, medical condition or treatment, and an individual subject's genetic makeup. Additionally, agents that are typically used for other purposes may possess local anesthetic or membrane stabilizing properties under certain circumstances or under particular conditions.

As used herein and in the claims, the term “immunogen” refers to any agent that elicits an immune response. Examples of an immunogen include but are not limited to natural or synthetic (including modified) peptides, proteins, lipids, oligonucleotides (RNA, DNA, etc.), chemicals, or other agents.

As used herein and in the claims, the term “allergen” refers to any agent that elicits an allergic response. Some examples of allergens include but are not limited to chemicals and plants, drugs (such as antibiotics, serums), foods (such as milk, wheat, eggs, etc), bacteria, viruses, other parasites, inhalants (dust, pollen, perfume, smoke), and/or physical agents (heat, light, friction, radiation). As used herein, an allergen may be an immunogen.

As used herein and in the claims, the term “adjuvant” and any derivation thereof refers to an agent that modifies the effect of another agent while having few, if any, direct effects when given by itself. For example, an adjuvant may increase the potency or efficacy of a pharmaceutical, or an adjuvant may alter or affect an immune response.

As used herein and in the claims, the term “opioid” generally refers to any agent that binds to and/or interacts with opioid receptors. Among the opioid classes examples include endogenous opioid peptides, opium alkaloids (e.g., morphine, codeine, and the like), semi-synthetic opioids (e.g., heroin, oxycodone and the like), synthetic opioids (e.g., buprenorphinemeperidine, fentanyl, morphinan, benzomorphan derivatives, and the like), as well as opioids that have structures unrelated to the opium alkaloids (e.g., pethidine, methadone, and the like).

As used herein and in the claims, the terms “vehicle,” “carrier,” “pharmaceutical vehicle,” “pharmaceutical carrier,” “pharmaceutically acceptable vehicle,” or “pharmaceutically acceptable carrier” may be used interchangeably, and refer to pharmaceutically acceptable solid or liquid, diluting or encapsulating, filling or carrying agents, which are usually employed in the pharmaceutical industry for making pharmaceutical compositions. Examples of vehicles include any liquid, gel, salve, cream, solvent, diluent, fluid ointment base, vesicle, liposome, nisome, ethasomes, transfersome, virosome, non-ionic surfactant vesicle, phospholipid surfactant vesicle, micelle, and the like, that is suitable for use in contacting a subject.

In some embodiments, the pharmaceutical vehicle may refer to a composition that includes and/or delivers a pharmacologically active agent, but is generally considered to be otherwise pharmacologically inactive. In some other embodiments, the pharmaceutical vehicle may have some therapeutic effect when applied to a site such as a mucous membrane or skin, by providing, for example, protection to the site of application from conditions such as injury, further injury, or exposure to elements. Accordingly, in some embodiments, the pharmaceutical vehicle may be used for protection without a pharmacological agent in the formulation.

The headings provided herein are for convenience only and do not interpret the scope or meaning of the embodiments.

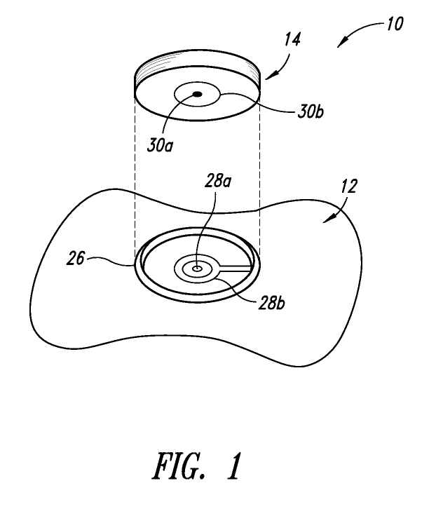

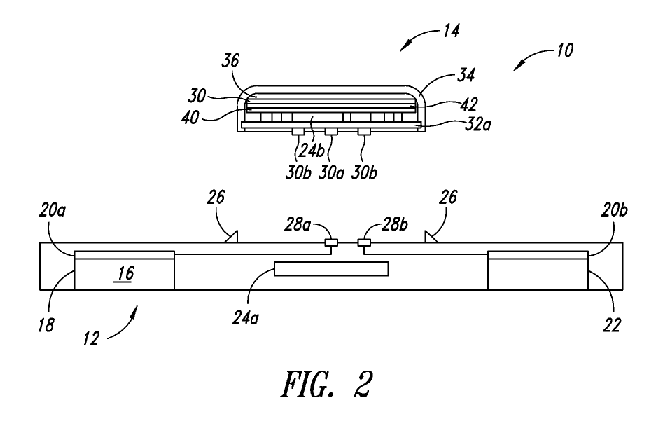

FIGS. 1 and 2 show an electrically powered or active transdermal delivery device 10 (e.g., an iontophoretic, electroporation, electrophoresis, etc. type delivery device) according to one illustrated embodiment.

The electrically powered or active transdermal delivery device 10 includes two distinct portions, an active agent delivery component 12 and a portable power supply system 14 that is selectively coupleable to provide power to the active agent delivery component 12 and selectively decoupleable therefrom. This electro-mechanical interface separates the electronics (i.e., portable power supply system 14) from the transdermal patch (i.e., active agent delivery component 12) so that each may be manufactured separately.

The active agent delivery component 12 stores or otherwise carries one or more active agents 16 (FIG. 2) stored in one or more active agent reservoirs 18 (FIG. 2) which may provide a therapeutic effect in a biological subject. The active agent 16 may be delivered, or delivery may be enhanced, through the use of an electrical potential applied via one or more electrodes 20a (FIG. 2) of the active agent delivery component 12. The active agent delivery component 12 may, for example, take the form of a patch. The active agent delivery component 12 may include one or more counter reservoirs 22 (FIG. 2) and/or counter electrodes 20b (FIG. 2) receive ions with an opposite polarity as that of the active agent 16. The active agent delivery component 12 is typically placed in direct or indirect contact on a biological interface (e.g., skin, mucus membrane) of a biological entity. Hence, the active agent delivery component 12 is typically disposed of after a single use.

The active agent delivery component 12 includes at least one coupler element 24a (FIG. 2) of a magnetic coupler (collectively 24). The coupler element 24a may take the form of a magnet or a ferrous element.

The active agent delivery component 12 may include a positioning structure 26 that facilitates the correct positioning of the portable power supply system 14 on the active agent delivery component 12. Such may, for example, take the form of a ridge or lip.

The active agent delivery component 12 may include one or more electrical coupling structures 28a, 28b (collectively 28) that allow electrical connections to be made with respective electrical coupling structures 30a, 30b (collectively 30) of the portable power supply system 14. Examples of electrical coupling structures 28, 30 may include one or more contacts, leads, terminals, polarized coupling elements, multi-pin connectors, DIN connectors, polarized multi-pin connectors, circular connectors, slot type interconnectors, inductors, plates, and the like, which are or may be positioned with respect to one another to effectively transfer power between the portable power supply system 10 and the active agent delivery component 12. Transfer of power may, for example, be electrically, conductively or capacitively, or may be inductively. Where inductive, each of the portable power supply system 10 and the active agent delivery component 12 may carry one or more windings (e.g., a primary and a secondary, respectively), that may be position with respect to one another to inductively transfer power to active agent delivery component 12 from the portable power supply system 10. In such an embodiment, the portable power supply system 10 may include an inverter (e.g., a switch mode inverter) configured to convert a DC current from the power source 30 into an alternating current to supply to the windings. In such an embodiment, the active agent delivery component 12 may include a rectifier (e.g., diode bridge) configured to rectify the AC current into a DC current suitable for applying the desired electrical potential at the electrodes.

The electrical coupling structures 28a, 28b may, for example, include an electrically positive and an electrically negative contact, and the electrical coupling structures 30a, 30b may likewise include an electrically positive and an electrically negative contact. In some embodiments, the spacing between and/or geometry of the electrical coupling structures 28, 30 precludes shorting when the portable power supply system 10 is physically coupled to the active agent delivery component 12 by the magnetic coupler 24. For example, in the illustrated embodiment, the electrical coupling structures 28, 30 take the form of electrical traces or contacts, which form a concentric geometric pattern that provides universally oriented proper electrical polarity alignment of the electrical coupling structures 28, 30. Additionally, the concentric pattern formed by the pairs of electrical coupling structures 28, 30 may create a visual target (e.g., “bulls-eye”) for a user when coupling the portable power supply system 10 to the active agent delivery component 12.

The portable power supply system 10 may include one or more coupler elements 24b to physically magnetically couple the portable power supply system 10 to the active agent delivery component 12.

The coupler elements 24a, 24b of the magnetic coupler 24 may take a variety of forms, for instance, one or more permanent magnets, one or more ferrous paramagnetic materials, one or more ferrous, ferromagnetic, or ferrimagnetic elements or coatings and/or one or more electromagnets. Permanent magnets may include high-energy flexible magnets, neodymium magnets, ceramic magnets, samarium cobalt magnets, alnico magnets, rare earth magnets, and the like. Paramagnetic materials (e.g., aluminum, copper, lithium, magnesium, molybdenum, platinum, tantalum, and the like) typically have a small and positive susceptibility (a relative magnetic permeability greater than unity) to magnetic fields and are attracted to a magnetic field (e.g., a magnet). Ferromagnetic materials (e.g., cobalt, iron, nickel, gadolinium, steel, and the like) typically have a large and positive susceptibility to magnetic fields and are attracted to a magnetic field. The coupler elements 24a, 24b should be complementary to one another. That is the elements 24a, 24b should be capable of retaining the portable power supply system 14 to the active agent delivery component 12, for example during normal use. For instance, the coupler elements 24a, 24b may be magnets of opposite magnetic polarities or may be a magnet and a ferrous material.

Thus, in some embodiments the electrically powered or active transdermal delivery device 10 may employ two concentric electrode contacts on the mating surfaces of the power supply (portable power supply system 10) and the transdermal patch (active agent delivery component 12) and co-axial placement of rare earth magnets in both the power supply and the transdermal patch. The magnets align the mating parts and provide the holding force while the concentric contacts eliminate any need to orient the power supply relative to the transdermal patch. The result is a simple, easy to use solution.

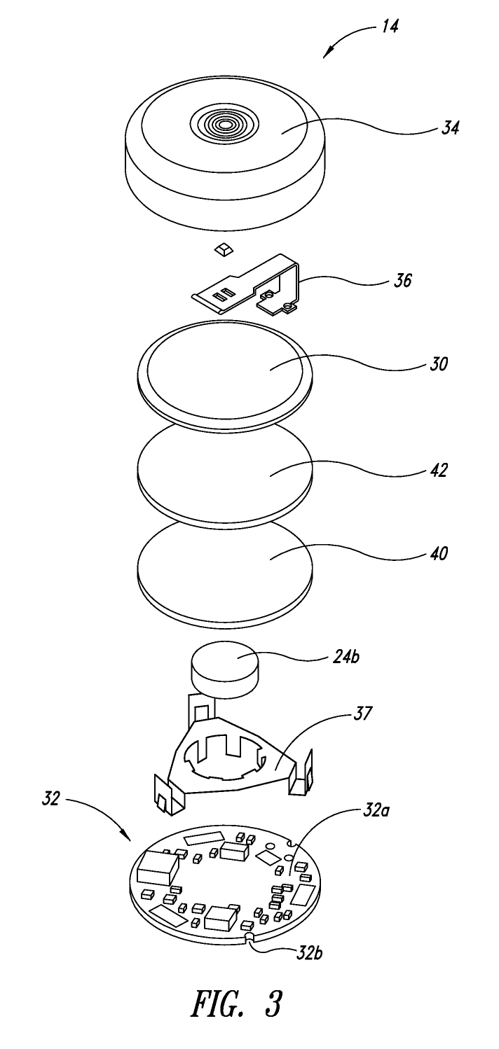

Various elements of the portable power supply system 14 are best illustrated in FIG. 3, according to one illustrated embodiment.

As noted above, the portable power supply system 14 includes at least one coupler element 24b of a magnetic coupler 24. Some embodiments may employ two or more coupler elements 24b (e.g., of opposite magnetic polarities) on the portable power supply system 14, which may, for example, ensure correct electrical polarity between the electrical coupling structures 28, 30. The magnetic coupler 24 allows the portable power supply system 14 to be selectively magnetically releasably attached and/or selectively magnetically releasably coupled to the active agent delivery component 12. In some embodiments, the coupler elements 24a, 24b may by electrically conductive and also function as the electrical coupling structures 28, 30, respectively.

The portable power supply system 14 also includes a power source 30. The power source 30 may take a variety of forms, for example one or more battery cells, super- or ultra-capacitors or fuel cells. For example, the power source 30 may take the form of at least one primary cell or secondary cell. Also for example of the power source 30 may take the form of a button cell, a coin cell, an alkaline cell, a lithium cell, a lithium ion cell, a zinc air cell, a nickel metal hydride cell, and the like. In some embodiments, the power source 30 takes the form of at least one printed battery, energy cell laminate, thin-film battery, power paper, and the like, or combinations thereof.

The portable power supply system 14 may further include a circuit 32 which may be carried by (i.e., on or in) a circuit board 32a. The circuit electrically couples the power source 30 to the electrical coupling structures 30a, 30b. The circuit may additionally be configured to perform one or more functions (e.g., voltage and/or current regulation, monitoring) as explained in further detail below.

The power supply system 14 may optionally include a cover 34 that may provide environmental protection to the various other elements of the power supply system 14, as well as provide protection (e.g., electrical and/or thermal insulation) to the biological subject.

The power supply system 14 may optionally include a power source holder 36. Such may facilitate manufacturing, allowing the power source 30 to be easily electrically coupled to the circuit 32. In some embodiments, the power source holder 36 may allow the power source 30 to be replaced, for example when insufficient charge remains. In such embodiments, the circuit board 32a may be detachably affixed to the cover 34 to allow access to an interior thereof to replace the power source 30 in the power source holder 36. For example, the circuit board 32a may be detachably affixed to the cover 34 using one or more couplers, fasteners, friction-fit structures, thread-coupled structures, bayonet-coupled structures, lip or rim in groove, and the like. The circuit board 32a may include a notch 32b (only one called out in FIG. 3) to facilitate the prying or disengaging of the circuit board 32a from the cover 34. In some embodiments, circuit board 32 may include a tab (not shown) to facilitate the prying or disengaging of circuit board 32 from the cover 34.

The power supply system 14 may optionally include a coupler element holder 37. Such may facilitate manufacturing, allowing the coupler element 24b (e.g., magnet) to be easily physically coupled to the circuit 32.

The power supply system 14 advantageously includes a magnetic flux blocker 40. The magnetic flux blocker 40 may, for example, take the form of a ferrous element, for example a disc of ferrous material. The magnetic flux blocker 40 is positioned between the coupler element 24b and the power source 30, to prevent magnetic flux from adversely affecting the power source 30. The magnetic flux blocker 40 may have a diameter or other lateral dimension that is at least equal to or greater than a corresponding lateral dimension of the power source 30 or the coupler element 24b. The magnetic flux blocker 40 may have a thickness or other longitudinal dimension that provides sufficient blocking or diverting of the magnetic flux to prevent adverse affects on the power source 30.

The power supply system 14 optionally includes a spacer 42 received between the magnetic flux blocker 40 and the power source 30. The spacer 42 may increase the distance between the coupler element 24b and the power source 30, thereby additionally reducing any adverse affect of magnetic flux on the power source 30. The spacer 42 may have a diameter or other lateral dimension that is at least equal to or greater than a corresponding lateral dimension of the power source 30 or the coupler element 24b. The spacer 42 may have a thickness or other longitudinal dimension that provides sufficient spacing of alleviated the adverse affects of magnetic flux on the power source 30.

During the development of the power supply, prototypes exhibited a limited shelf life. Instead of an expected two or more years, the shelf life of power supplies having a chemical battery power source rarely exceeded 2 weeks. A series of experiments showed that this degradation in performance was attributable to the presence of strong magnetic fields in close proximity with the chemical battery power source. Subsequent inquires with battery manufacturers demonstrated that they had little or no experience with the exposure of their batteries to magnetic fields and gave no credence to the concept of battery degradation when exposed to magnetic fields. A series of experiments demonstrated that: 1) chemical batteries exposed to magnetic fields similar to those found in the iontophoresis power supply for greater than 2 weeks show noticeable degradation in both battery voltage and battery capacity; and 2) use of magnetically permeable materials can steer the magnetic flux away from the battery and thereby reduce the extent of the degradation.

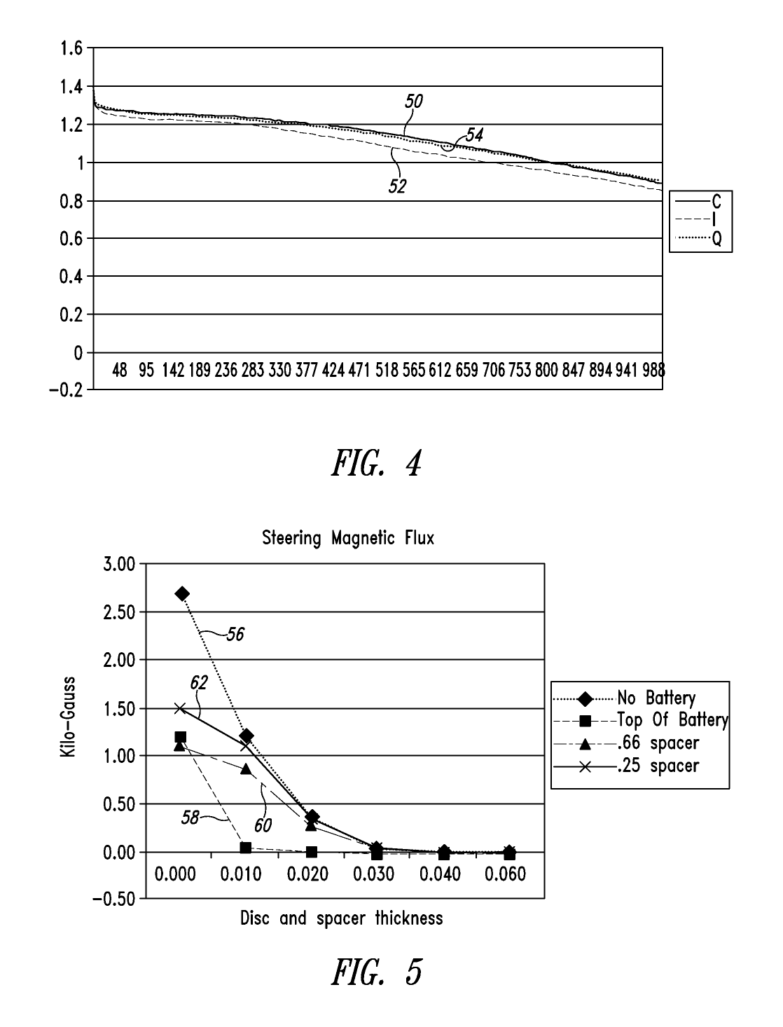

FIG. 4 is a graph showing battery discharge performance of several power supply systems after 2 weeks quiescent current. A first curve 50 indicates the battery discharge performance of a power supply system having a chemical battery but without a magnet. A second curve 52 indicates the battery discharge performance of a power supply system having a chemical battery and an adjacent magnetic coupler element, but without a magnetic field blocker and spacer. A third curve 54 indicates the battery discharge performance of a power supply system having chemical battery and a magnetic coupler element, and which includes a magnetic field blocker and spacer positioned between the chemical battery and magnetic coupler element. Notably, the third curve 54 (with magnetic field blocker and spacer) closely follows the first curve 50 (no magnet present) and shows significantly less adverse affect than the second curve 52 (magnet present but no magnetic field blocker or spacer). Thus, a magnetic field blocker and spacer can reduce or eliminate the adverse affect that the magnetic field of a magnet has on a chemical battery. Without being limited by theory, it is believed that the magnetic field blocker turns or “steers” the magnetic field or flux away from the chemical battery. It is also believed that the spacer reduces the strength of any magnetic field on the chemical battery.

Table 1 summarizes tests conducted by using a Gauss meter to measure the magnetic field strength for different physical arrangements. Adequate results were achieved using a 0.030 inch thick ferrous disc as the magnetic blocker, however a thickness of 0.040 inch (1 mm) was chosen for the product. The rationale for choosing this more conservative solution is that: a) 1 mm is a standard material thickness that is readily available; b) it may be preferable to use a single part to achieve the desired results; and c) if necessary, it is possible to fall back to a disc thickness of 0.030 inches plus an inert spacer of 0.010 inches without impacting the tooling for the rest of the device.

TABLE 1

| | | | | | |
|---|---|---|---|---|---|
| Ambient | −0.08 | | | | |
| Magnet alone | 2.70 | | | | |
| Top of battery | 1.20 | | | | |
| Shim thickness | 0.060 | 0.040 | 0.030 | 0.020 | 0.010 |
| No Battery | 0.00 | 0.00 | 0.03 | 0.36 | 1.20 |
| Top Of Battery | −0.01 | −0.01 | −0.01 | 0.00 | 0.03 |
| .66 spacer | 0.00 | 0.00 | 0.02 | 0.28 | 0.87 |
| .25 spacer | 0.00 | 0.00 | 0.03 | 0.32 | 1.10 |

FIG. 5 shows a graph of magnetic flux relative to magnetic field blocker thickness for various thicknesses of magnetic field blocker and spacer.

A first curve 56 represents a control, shows steering of magnetic flux where no battery is present. A second curve 58 shows the affect of a magnetic field blocker by thickness without a spacer present. A third curve 60 shows the affect of a magnetic field blocker by thickness with a spacer having a thickness of 0.66 inches present. A fourth curve 62 shows the affect of a magnetic field blocker by thickness with a spacer having a thickness of 0.25 inches present.

While described with the coupler element 24b being a magnet, the magnetic field blocker and spacer may also be advantageously effective where the magnet is the coupler element 24a carried by the active agent delivery component 12.

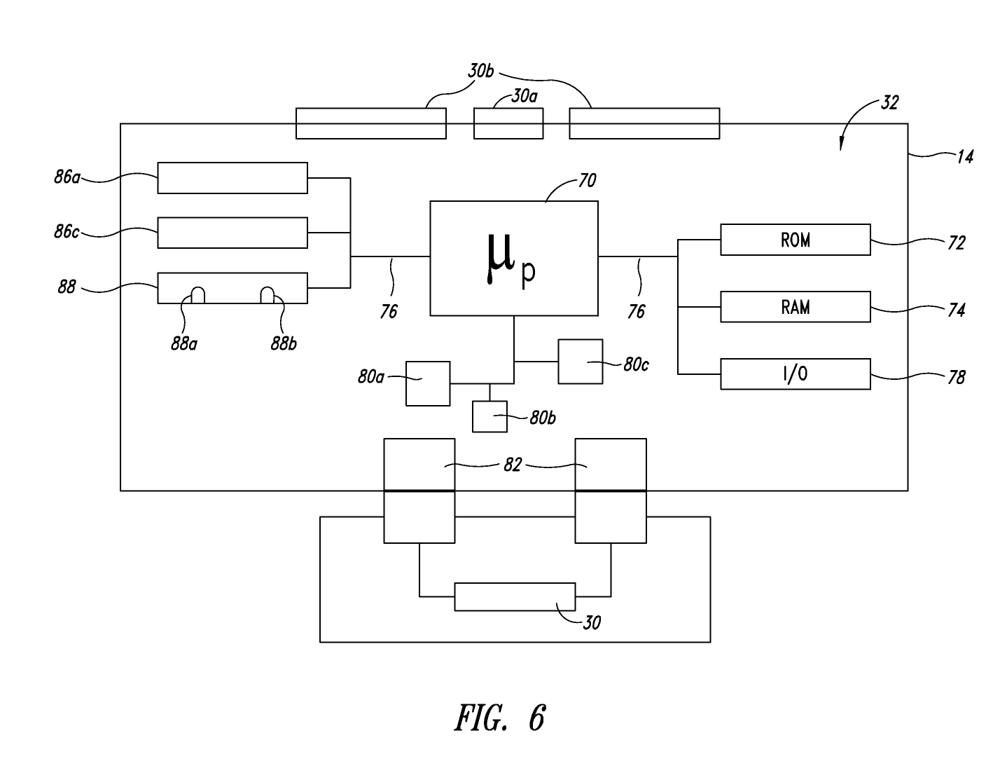

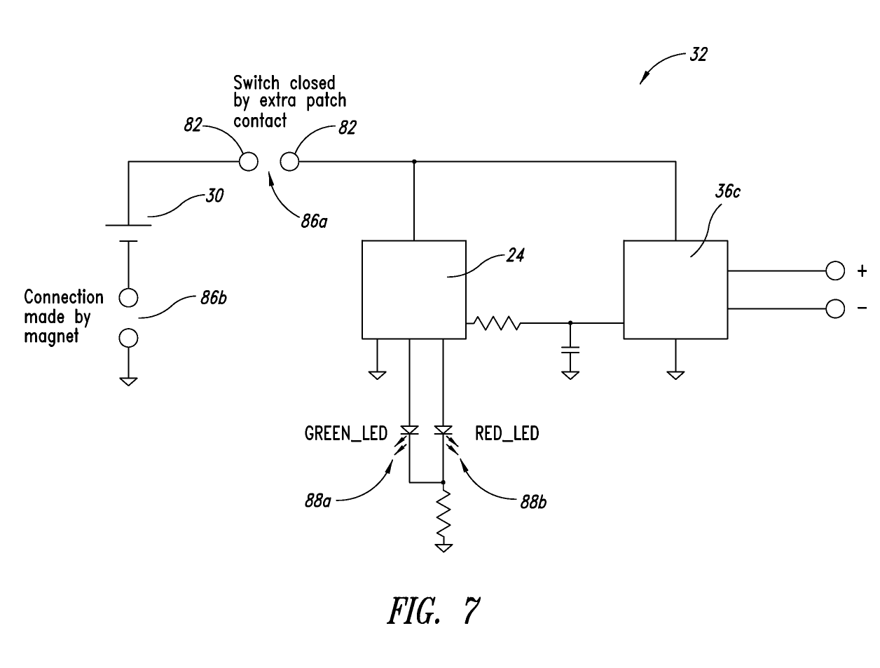

FIGS. 6 and 7 show a portable power supply system 14 according to one illustrated embodiment.

The portable power supply system 14 may include a control system in the form of a control circuit 32 to control the voltage, current, and/or power delivered to the active agent delivery component 12 (FIGS. 1 and 2). The control circuit 32 may include one or more controllers 70 such as a microprocessor, a digital signal processor (DSP) (not shown), an application-specific integrated circuit (ASIC) (not shown), field programmable gate array (FPGA) and the like. The control circuit 32 may also include one or more memories, for example, read-only memory (ROM) 72, random access memory (RAM) 74, and the like, coupled to the controller(s) 70 by one or more busses 76 (e.g., instructions bus, data bus, power bus, etc.). The control circuit 32 may further include one or more input devices 78 (e.g., a display, touch-screen display, keys, buttons, LCDs, LEDs, and the like).

The control circuit 32 may also include discrete and/or integrated circuit elements 80a, 80b, 80c to control the voltage, current, and/or power. For example, the control circuit 32 may include a diode to provide a constant current to electrodes of the active agent delivery component 12 (FIGS. 1 and 2). In some embodiments, the control circuit 32 may include a rectifying circuit element to provide a direct current voltage and/or to function as a voltage/current regulator. In other embodiments, the control circuit 32 sinks and sources voltage to maintain a steady state operation of the active agent delivery component 12 (FIGS. 1 and 2). The control circuit 32 may be electrically coupled to receive current from the power source 30, via electrical contacts 82. In some embodiments, the control circuit 32 may take the form of a programmable control circuit operable to provide at least a first current profile. In some embodiments, the control circuit 32 may take the form of a programmable control circuit operable to provide a plurality of current profiles. For example, the control circuit 32 may be operable to provide at least a first current profile associated with the control delivery, sustained delivery, and the like, associated with transdermal delivery of one or more active agents to a biological interface of a subject. In some embodiments, the portable power supply system 14 is operable to provide a current ranging from about 10 mA·min to about 80 mA·min for a period ranging from about 1 min to about 24 hrs.

In some embodiments, the control circuit 32 is configured to track, store, transmit, receive, and/or retrieve treatment management data. For example, the control circuit 32 may be configured to track, store, transmit, receive, and/or retrieve transdermal delivery device information. In some embodiments, the control circuit 32 may be configured to query tag data (e.g., a Radio Frequency Identification (RFID) tag) including for example stored data codes, user data, patient data, drug delivery device data, and the like.

In some embodiments, the control circuit 32 is configured to store and/or track historical data, use data, patient data, and the like. In some embodiments, the control circuit 32 includes an RFID type chip to store, track, receive, and retrieve delivery device (e.g., iontophoretic delivery device, transdermal patch, and the like) information, query tag data, store data codes, track use data, track patient data, and the like. The RFID type chip may take the form of, for example, an active type RFID type chip, receiving power form the portable power supply system 14. In some embodiments, the RFID type chip may take the form of a passive type RFID type chip using, for example, only a memory portion of RFID type chip. In some embodiments, a portion of the RFID type chip is used for memory without using the RF capabilities of the RFID type chip. Such may advantageously take advantage of low cost chips produced for high volume applications such as RFID.

As shown in FIG. 7, in some embodiments, the control circuit 32 is configured to automatically close in response to the portable power supply system 14 being releasably attached to the active agent delivery component 12. For example, the control circuit 32 may include a switch 86a that closes on completion of a circuit between the portable power supply system 14 and the active agent delivery component 12. In some embodiments, the control circuit 32 may additionally or alternatively include a magnetically response switch 86b that automatically closes (i.e., completes the circuit) in response to the coupler element 24a of the active agent delivery component 12 being proximate. The control circuit 32 may further include a start and/or stop switch 86c operable to selectively control the flow of current to the control circuit 32. The switch 86c may take the form of a dome switch, a membrane switch, a tactile switch, a single-use dome switch, a single-use membrane switch, a single-use tactile switch, and the like.

In some embodiments, the control circuit 32 may include self-test capabilities that are initiated once the control circuit 32 is closed.

In some embodiments, the control circuit 32 may be operable to detect a power source polarity and provide a charge of a proper polarity to respective ones of a positive electrical contact and a negative electrical contact of the active agent delivery component 12, in response to the portable power supply system 14 being releasably attached to the active agent delivery component 12.

The portable power supply system 14 may further include one or more indicators, collectively 88, to, for example, alert a user that the portable power supply system 14 is operating properly. Examples of the one or more indicators 88 include visual feedback elements 88a, 88b (e.g., light-emitting diodes or LEDs such as green and red LEDs, a display, and the like). The indicators 88 may additionally or alternatively include audio feedback elements (not shown) (e.g., a speaker) and/or tactile feedback elements, and the like.

In some embodiments, the portable power supply system 14 has a largest dimension of less than about 25 mm, and a smallest dimension of less than about 10 mm. In some embodiments, the portable power supply system 14 has an aspect ratio ranging from about 2:1 to about 13:1.

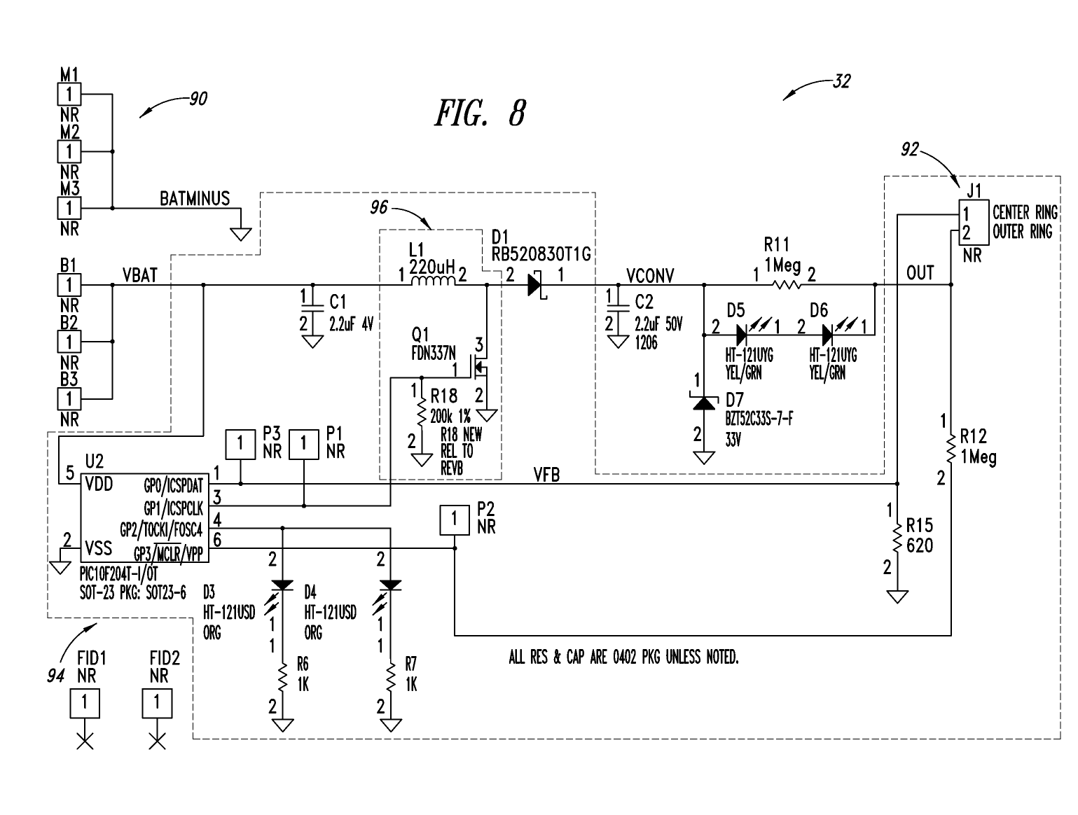

FIG. 8 shows a control circuit 32, according to one illustrated embodiment.

The control circuit 32 includes a number of input terminals 90, a number of output terminals 92, a regulation circuit 94 coupled between the input and output terminals 90, 92, a number of indicators D3-D6, and a controller U2.

The input terminals 90 provide a structure to electrically couple the control circuit 32 to a power source 30 (FIGS. 6 and 7), for example a chemical battery cell. As illustrated, the control circuit 32 may include three input terminals B1-B3 of a first polarity and three input terminals M1-M3 of a second polarity. Such may ensure good electrical contact with the power source 30, although some embodiments may employ a lesser or greater number of input terminals.

The output terminals 92 provide a structure to supply electrical power to electrodes of an active agent delivery device 12 (FIGS. 1 and 2), for example an iontophoresis patch. As noted previously, the output terminals 92 may comprise a first and a second terminal, one for each polarity. The output terminals 92 may be configured, shaped, and/or arranged to assure that the correct polarity is maintained when coupling, for example, the portable power supply system 14 to the active agent delivery component 12. For example, the output terminals 92 may be formed as two concentric structures, for example, an inner pad and an outer annulus or ring surrounding the inner pad 28a, 28b (FIG. 1). In some embodiments, the inner pad may take the form of an annulus or ring shape, however other shapes are possible. Further, in some embodiments, the other structure may be a shape other than an annulus or ring. In some embodiments, one of the output terminals 92 may substantially or completely surround in the other output terminal 92, while in other embodiments, one of the output terminals 92 may only partially surround or may not even partially surround the other output terminal 92.

The regulation circuit 94 includes a power converter 96 operable to adjust or maintain a current and/or voltage at the output terminals 92, for example, as discussed in detail below. The power converter 96 may take the form of current regulator, boost converter, buck converter, or some combination of the same, for instance a switch mode power converter. As illustrated, the regulation circuit 94 may take the form of a boost converter formed, for example, by an inductor L1 coupled between the input and output terminals of a switch Q1 operable to selectively couple the inductor L1 to ground. In the embodiment illustrated in FIG. 8, the switch Q1 may take the form of a transistor having a gate, a drain, and a source.

The control circuit 32 may include a Schottky diode D1 to prevent damage in the event of a reversal of polarity. The control circuit 32 may include a Zener diode D7 coupled to ground to prevent output voltage from exceeding a desired level. The control circuit 32 may also include an input capacitor C1 coupled to ground, between the output terminals 92 and the inductor L1 to act as an input filter. The control circuit 32 may further include an output capacitor C2 coupled to ground, between the inductor L1 and the output terminals 92 to act as an output filter, reducing ripple in the output current.

The controller U2 may take a variety of forms, for example a microcontroller, processor, microprocessor, digital signal processor, field programmable gate array, or the like. The controller U2 includes power and ground connectors or pins VDD, VSS. The controller U2 supplies drive signals to the gate of the transistor Q1 via output or pin 3 of the controller U2. Inputs or pins 1, 6 of the controller U2 are coupled to terminals 1, 2 of the output terminals 92, respectively. Thus, the controller U2 can determine or sense the operating characteristics at the output terminals 92. For example, the controller U2 may be responsive to the presence or absence of a load across the output terminals 92 via a load sense resistor R12. The value of R12 may be selected such that the impedance associated with skin or other biological tissue (e.g., 20K Ohms) is sufficient to trigger the controller U2. The controller U2 may also be responsive to a measure of voltage across and/or flow of current at the output terminals 92. For example, in the illustrated embodiment the controller U2 is responsive to a measure of current via a current sense resistor R15.

The controller U2 may be normally powered and/or in the ON state, and may be programmed or otherwise configured to perform certain functions upon detection or in response to a load across the output terminals 92 via load sense resistor R12. For example, the controller U2 may be configured or programmed to perform one or more tests, provide appropriate indications, and/or take appropriate actions based on the results of the tests, and/or start delivery active agent according to one or more delivery profiles. For example, the controller U2 may enter a test or startup mode upon detection of a load, and may enter a current supply mode upon successful completion of the test and startup mode. The controller U2 may be in a wait or sleep mode prior to detecting a load, for example, while the portable power supply system 14 is in storage. An energy efficient controller U2 may be stored for many years while monitoring for the presence of the load, particularly where the power source 30 (FIGS. 2, 3, 6 and 7) is protected from magnetic flux of the coupler element 24b (FIGS. 2 and 3).

The controller U2 may be programmed or otherwise configured to employ a measure of voltage across, or current through, the output terminals 92 to maintain a desired delivery profile, for example, a constant current delivery profile. In one embodiment, the controller U2 may provide drive signals to maintain a constant, or approximately constant, current output at the output terminals 92 over at least a portion of a delivery profile. In one embodiment, the controller U2 may provide drive signals to provide an increasing current output at the output terminals 92 over at least a portion of a delivery profile. For example, the controller U2 may provide drive signals to produce an increasing current over an initial portion of a delivery profile. The increasing current may increase linearly or nonlinearly. Also for example, the controller U2 may provide drive signals to produce an increasing current over a terminal portion of a delivery profile. In one embodiment, the controller U2 may provide drive signals to provide a varying current output at the output terminals 92 over at least a portion of a delivery profile. For example, the controller U2 may provide drive signals to produce a varying current over an initial portion of a delivery profile, terminal portion of a delivery profile or some intermediate portion of a delivery profile. The current may vary periodically, for example sinusoidally, or may vary aperiodically. In the illustrated embodiment, the controller U2 sets a duty cycle of a drive signal supplied to the gate of the transistor Q1 in order to maintain a constant current output at the output terminals 92. In particular, the controller U2 may start with a low duty cycle, increasing the duty cycle until a voltage supplied at pin 1 via the current sense resistor R15 matches a reference voltage V_{ref}. The reference voltage V_{ref} may be stored or defined internally in the controller U2, and may, for example be approximately 0.6V. The controller U2 may oscillate, or vary, the duty cycle to maintain the desired constant current operation.

In the embodiment illustrated, the indicators D5, D6 may take the form of two or more light emitting diodes (LEDs) D5, D6 electrically coupled in series. The two or more LEDs D5, D6 may be electrically coupled in parallel with a resistor R11. The LEDs D5, D6 may both produce the same color(s), when driven to emission, for example green (or green light). When lit, the LEDs D5, D6 indicate that current is flowing to the output terminals.

In the embodiment illustrated, the indicators D3, D4 may take the form of LEDs D3, D4. The LED D3 and the LED D4 are each electrically coupled between an output of the controller U2, and ground through respective resistors R6, R7. The LEDs D3, D4 may both produce the same color(s), when driven to emission. The LEDs D3, D4 may produce, for example, a color different from the color produced by the LEDs D5, D6. For example, the LEDs D3, D4 may produce red or orange light. The LEDs D3, D4 may provide a first indication during start up, or to indicate a proper start up (e.g., blinking a predetermined number of times). The LEDs D3, D4 may provide a first indication during shut down, to indicate the delivery profile is terminating or has been terminated (e.g., blinking at a predetermined rate).

In some embodiments, the controller U2 may be programmed or otherwise configured to measure at least one of a voltage, current, resistance, impedance, and the like, indicative of, for example, a delivery device type (e.g., iontophoretic delivery device type, transdermal patch type, drug delivery device, and the like), a drug type, a dosing regimen, and the like. The controller U2 may be further configured to adjust a delivery profile, for example, a current delivery profile based on the measure of at least one of a voltage, a current, a resistance, an impedance, and the like. For example, the controller U2 may be programmed or otherwise configured to query the electrically powered device 11 and based on the response of the query, adjust a delivery profile, for example, a current delivery profile.

The control circuit 32 may take the form of a printed circuit on a substrate, for example a circuit board 32a (FIG. 3). Conductive paths may take the form of conductive traces forming a generally concentric geometric pattern. In certain embodiments, the conductive traces may be deposited, etched, or otherwise applied to the circuit board 32a. The conductive trace can comprise any suitable material for making a conductive trace including conductive polymers, metallic materials, copper, gold, silver, copper coated with silver or tin, aluminum, and/or alloys or combinations thereof. Techniques for making the circuit 32 on a circuit board 32 are well known in the art and include lithographic techniques, conductive paint silk screen techniques, metal deposition, conventional pattering techniques, laser etching, and the like. For example, well-known lithographic techniques can be use to form a conductive trace layout, onto at least the first surface of the circuit board 32a. The lithographic process for forming the conductive trace layout may include, for example, applying a resist film (e.g., spin-coating a photoresist film) onto the substrate, exposing the resist with an image of a circuit layout (e.g., the geometric pattern of one or more conductive traces), heat treating the resist, developing the resist, transferring the layout onto the substrate, and removing the remaining resist. Transferring the layout onto the circuit board 32a may further include using techniques like subtractive transfer, etching, additive transfer, selective deposition, impurity doping, ion implantation, and the like. Some embodiments may employ flexible circuits, for instance single-sided flexible circuits, double-sided flexible circuits, multi-layered flexible circuits, adhesiveless flexible circuits, lightweight flexible circuits, rigid-flex circuits, and the like. In some embodiments, the flexible circuit may comprise a thin-film integrated circuit, for example a thin-film flexible circuit less than about 7000 μm thick and at least a portion of which is flexible about at least one bend axis. In some embodiments, the flexible circuit comprises one or more portions fabricated from conductive fabric.

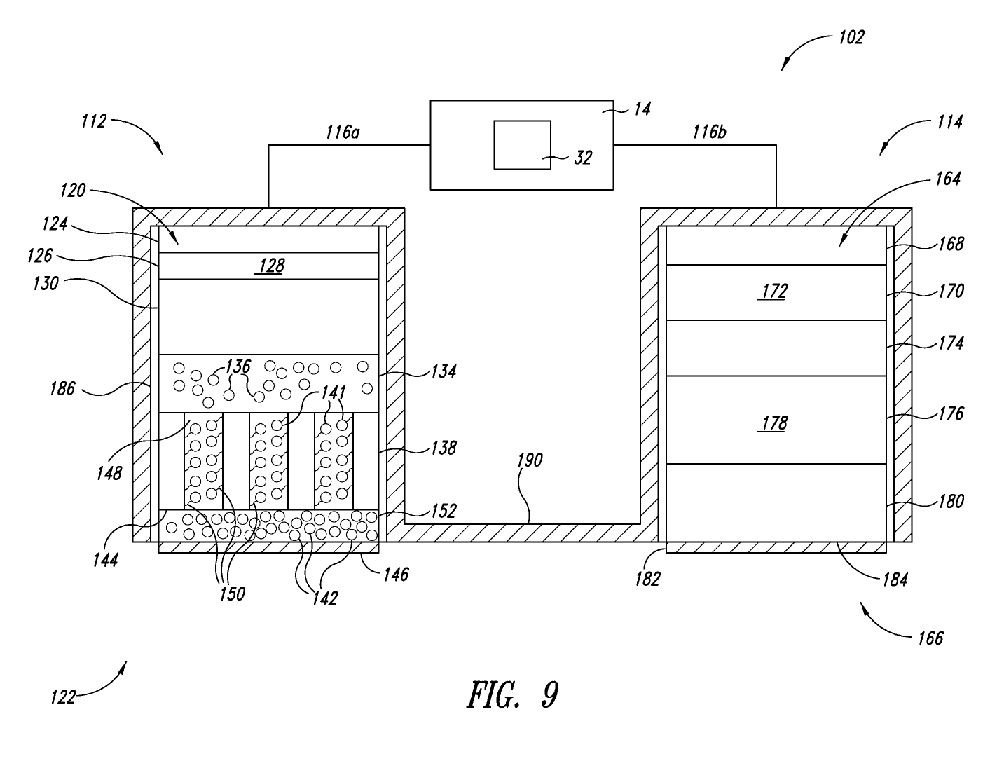

FIG. 9 shows an active transdermal deliver device 102 including an active agent delivery component 112 and a portable power supply system 14, according to one illustrated embodiment. The active agent delivery component 12 includes an active electrode assembly 112 and a passive electrode assembly 114, according to one illustrated embodiment.

The active electrode assembly 112 may comprise, from an interior 120 to an exterior 122 of the active electrode assembly 112: an active electrode element 124, an electrolyte reservoir 126 storing an electrolyte 128, an inner ion selective membrane 130, one or more inner active agent reservoirs 134, storing one or more active agents 136, an optional outermost ion selective membrane 138 that optionally caches additional active agents 141, and an optional further active agent 142 carried by an outer surface 144 of the outermost ion selective membrane 138. Each of the above elements or structures will be discussed in detail below.

The active electrode assembly 112 may comprise an optional inner sealing liner (not shown) between two layers of the active electrode assembly 112, for example, between the inner ion selective membrane 130 and the inner active agent reservoir 134. The inner sealing liner, if present, would be removed prior to application of the iontophoretic device to the biological surface 118. The active electrode assembly 112 may further comprise an optional outer release liner 146.

In some embodiments, the one or more active agent reservoirs 134 are loadable with a vehicle and/or pharmaceutical composition for transporting, delivering, encapsulating, and/or carrying the one or more active agents 136, 140, 142. In some embodiments, the pharmaceutical composition includes a therapeutically effective amount of the one or more active agents 136, 140, 142.

The active electrode element 124 is electrically coupleable via a first pole 116a to the portable power supply system 14 and positioned in the active electrode assembly 112 to apply an electromotive force to transport the active agent 136, 140, 142 via various other components of the active electrode assembly 112. Under ordinary use conditions, the magnitude of the applied electromotive force is generally that required to deliver the one or more active agents according to a therapeutic effective dosage protocol. In some embodiments, the magnitude is selected such that it meets or exceeds the ordinary use operating electrochemical potential of the transdermal delivery device 102. The at least one active electrode element 124 is operable to provide an electromotive force for driving a pharmaceutical composition comprising one or more active agents 136, 140, 142 from the at least one active agent reservoir 134, to the biological interface 118 of the subject.

The active electrode element 124 may take a variety of forms. In one embodiment, the active electrode element 124 may advantageously take the form of a carbon-based active electrode element. Such may comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004. The carbon-based electrodes are inert electrodes in that they do not themselves undergo or participate in electrochemical reactions. Thus, an inert electrode distributes current through the oxidation or reduction of a chemical species capable of accepting or donating an electron at the potential applied to the system, (e.g., generating ions by either reduction or oxidation of water). Additional examples of inert electrodes include stainless steel, gold, platinum, capacitive carbon, or graphite.

Alternatively, an active electrode of sacrificial conductive material, such as a chemical compound or amalgam, may also be used. A sacrificial electrode does not cause electrolysis of water, but would itself be oxidized or reduced. Typically, for an anode a metal/metal salt may be employed. In such case, the metal would oxidize to metal ions, which would then be precipitated as an insoluble salt. An example of such anode includes an Ag/AgCl electrode. The reverse reaction takes place at the cathode in which the metal ion is reduced and the corresponding anion is released from the surface of the electrode.

The electrolyte reservoir 126 may take a variety of forms including any structure capable of retaining electrolyte 128, and, in some embodiments, may even be the electrolyte 128 itself, for example, where the electrolyte 128 is in a gel, semi-solid or solid form. For example, the electrolyte reservoir 126 may take the form of a pouch or other receptacle, or a membrane with pores, cavities, or interstices, particularly where the electrolyte 128 is a liquid.

In one embodiment, the electrolyte 128 comprises ionic or ionizable components in an aqueous medium, which can act to conduct current towards or away from the active electrode element. Suitable electrolytes include, for example, aqueous solutions of salts. Preferably, the electrolyte 128 includes salts of physiological ions, such as sodium, potassium, chloride, and phosphate. In some embodiments, the one or more electrolyte reservoirs 124 include an electrolyte 128 comprising at least one biologically compatible anti-oxidant selected from ascorbate, fumarate, lactate, and malate, or salts thereof.

Once an electrical potential is applied, when an inert electrode element is in use, water is electrolyzed at both the active and counter electrode assemblies. In certain embodiments, such as when the active electrode assembly is an anode, water is oxidized. As a result, oxygen is removed from water while protons (H⁺) are produced. In one embodiment, the electrolyte 128 may further comprise an anti-oxidant. In some embodiments, the anti-oxidant is selected from anti-oxidants that have a lower potential than that of, for example, water. In such embodiments, the selected anti-oxidant is consumed rather than having the hydrolysis of water occur. In some further embodiments, an oxidized form of the anti-oxidant is used at the cathode and a reduced form of the anti-oxidant is used at the anode. Examples of biologically compatible anti-oxidants include, but are not limited to, ascorbic acid (vitamin C), tocopherol (vitamin E), or sodium citrate.

As noted above, the electrolyte 128 may take the form of an aqueous solution housed within a reservoir 126, or may take the form of a dispersion in a hydrogel or hydrophilic polymer capable of retaining a substantial amount of water. For instance, a suitable electrolyte may take the form of a solution of 0.5 M disodium fumarate: 0.5 M polyacrylic acid: 0.15 M anti-oxidant.

If included, the inner ion selective membrane 130 is generally positioned to separate the electrolyte 128 and the inner active agent reservoir 134. The inner ion selective membrane 130 may take the form of a charge selective membrane. For example, when the active agent 136, 140, 142 comprises a cationic active agent, the inner ion selective membrane 130 may take the form of an anion exchange membrane, selective to substantially pass anions and substantially block cations. The inner ion selective membrane 130 may advantageously prevent transfer of undesirable elements or compounds between the electrolyte 128 and the inner active agent reservoir 134. For example, the inner ion selective membrane 130 may prevent or inhibit the transfer of sodium (Na⁺) ions from the electrolyte 128, thereby increasing the transfer rate and/or biological compatibility of the transdermal delivery device 102.

The inner active agent reservoir 134 is generally positioned between the inner ion selective membrane 130 and the outermost ion selective membrane 138. The inner active agent reservoir 134 may take a variety of forms including any structure capable of temporarily retaining active agent 136. For example, the inner active agent reservoir 134 may take the form of a pouch or other receptacle, or a membrane with pores, cavities, or interstices, particularly where the active agent 136 is a liquid. The inner active agent reservoir 134 further may comprise a gel matrix.

Optionally, an outermost ion selective membrane 138 is positioned generally opposed across the active electrode assembly 112 from the active electrode element 124. The outermost membrane 138 may take the form of an ion exchange membrane having pores 148 of the ion selective membrane 138 including ion exchange material or groups 150. Under the influence of an electromotive force or current, the ion exchange material or groups 150 selectively substantially passes ions of the same polarity as active agent 136, 140, while substantially blocking ions of the opposite polarity. Thus, the outermost ion exchange membrane 138 is charge selective. Where the active agent 136, 140, 142 is a cation (e.g., lidocaine), the outermost ion selective membrane 138 may take the form of a cation exchange membrane, thus allowing the passage of the cationic active agent while blocking the back flux of the anions present in the biological interface, such as skin.

The outermost ion selective membrane 138 may optionally cache active agent 140. Without being limited by theory, the ion exchange groups or material 150 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 138 may take the form of a semi-permeable or microporous membrane that is selective by size. In some embodiments, such a semi-permeable membrane may advantageously cache active agent 140, for example by employing the removably releasable outer release liner to retain the active agent 140 until the outer release liner is removed prior to use.

The outermost ion selective membrane 138 may be optionally preloaded with the additional active agent 140, such as ionized or ionizable drugs or therapeutic agents and/or polarized or polarizable drugs or therapeutic agents. Where the outermost ion selective membrane 138 is an ion exchange membrane, a substantial amount of active agent 140 may bond to ion exchange groups 150 in the pores, cavities or interstices 148 of the outermost ion selective membrane 138.

The active agent 142 that fails to bond to the ion exchange groups of material 150 may adhere to the outer surface 144 of the outermost ion selective membrane 138 as the further active agent 142. Alternatively, or additionally, the further active agent 142 may be positively deposited on and/or adhered to at least a portion of the outer surface 144 of the outermost ion selective membrane 138, for example, by spraying, flooding, coating, electrostatically depositing, vapor depositioning, and/or otherwise. In some embodiments, the further active agent 142 may sufficiently cover the outer surface 144 and/or be of sufficient thickness to form a distinct layer 152. In other embodiments, the further active agent 142 may not be sufficient in volume, thickness, or coverage as to constitute a layer in a conventional sense of such term. The further active agent 142 may adhere to the outer surface 144 in the absence of electromotive force or current.

The active agent 142 may be deposited in a variety of highly concentrated forms such as, for example, solid form, nearly saturated solution form, or gel form. If in solid form, a source of hydration may be provided, either integrated into the active electrode assembly 112, or applied from the exterior thereof just prior to use.

In some embodiments, the active agent 136, additional active agent 140, and/or further active agent 142 may be identical or similar compositions or elements. In other embodiments, the active agent 136, additional active agent 140, and/or further active agent 142 may be different compositions or elements from one another. Thus, a first type of active agent may be stored in the inner active agent reservoir 134, while a second type of active agent may be cached in the outermost ion selective membrane 138. In such an embodiment, either the first type or the second type of active agent may be deposited on the outer surface 144 of the outermost ion selective membrane 138 as the further active agent 142. Alternatively, a mix of the first and the second types of active agent may be deposited on the outer surface 144 of the outermost ion selective membrane 138 as the further active agent 142. As a further alternative, a third type of active agent composition or element may be deposited on the outer surface 144 of the outermost ion selective membrane 138 as the further active agent 142. In another embodiment, a first type of active agent may be stored in the inner active agent reservoir 134 as the active agent 136 and cached in the outermost ion selective membrane 138 as the additional active agent 140, while a second type of active agent may be deposited on the outer surface 144 of the outermost ion selective membrane 138 as the further active agent 142. Typically, in embodiments where one or more different active agents are employed, the active agents 136, 140, 142 will all be of common polarity to prevent the active agents 136, 140, 142 from competing with one another. Other combinations are possible.

The outer release liner may generally be positioned overlying or covering further active agent 142 carried by the outer surface 144 of the outermost ion selective membrane 138. The outer release liner may protect the further active agent 142 and/or outermost ion selective membrane 138 during storage, prior to application of an electromotive force or current. The outer release liner may be a selectively releasable liner made of waterproof material, such as release liners commonly associated with pressure sensitive adhesives.

An interface-coupling medium (not shown) may be employed between the electrode assembly and the biological interface 118. The interface-coupling medium may take, for example, the form of an adhesive and/or gel. The gel may take the form of, for example, a hydrating gel. Selection of a suitable bioadhesive gels is within the knowledge of one skilled in the relevant art.

In the embodiment illustrated in FIG. 9, the counter electrode assembly 114 comprises, from an interior 164 to an exterior 166 of the counter electrode assembly 114: a counter electrode element 168, an electrolyte reservoir 170 storing an electrolyte 172, an inner ion selective membrane 174, an optional buffer reservoir 176 storing buffer material 178, an optional outermost ion selective membrane 180, and an optional outer release liner 182.

The counter electrode element 168 is electrically coupleable via a second pole 116b to the portable power supply system 14, the second pole 116b having an opposite polarity to the first pole 116a. In one embodiment, the counter electrode element 168 is an inert electrode. For example, the counter electrode element 168 may take the form of the carbon-based electrode element discussed above.

The electrolyte reservoir 170 may take a variety of forms including any structure capable of retaining electrolyte 172, and, in some embodiments, may even be the electrolyte 172 itself, for example, where the electrolyte 172 is in a gel, semi-solid or solid form. For example, the electrolyte reservoir 170 may take the form of a pouch or other receptacle, or a membrane with pores, cavities, or interstices, particularly where the electrolyte 172 is a liquid.

The electrolyte 172 is generally positioned between the counter electrode element 168 and the outermost ion selective membrane 180, proximate the counter electrode element 168. As described above, the electrolyte 172 may provide ions or donate charges to prevent or inhibit the formation of gas bubbles (e.g., hydrogen or oxygen, depending on the polarity of the electrode) on the counter electrode element 168 and may prevent or inhibit the formation of acids or bases or neutralize the same, which may enhance efficiency and/or reduce the potential for irritation of the biological interface 118.

The inner ion selective membrane 174 may be positioned between the electrolyte 172 and the buffer material 178. The inner ion selective membrane 174 may take the form of a charge selective membrane, such as the illustrated ion exchange membrane that substantially allows passage of ions of a first polarity or charge while substantially blocking passage of ions or charge of a second, opposite polarity. The inner ion selective membrane 174 will typically pass ions of opposite polarity or charge to those passed by the outermost ion selective membrane 180 while substantially blocking ions of like polarity or charge. Alternatively, the inner ion selective membrane 174 may take the form of a semi-permeable or microporous membrane that is selective based on size.

The inner ion selective membrane 174 may prevent transfer of undesirable elements or compounds into the buffer material 178. For example, the inner ion selective membrane 174 may prevent or inhibit the transfer of hydroxy (OH⁻) or chloride (Cl⁻) ions from the electrolyte 172 into the buffer material 178.

The optional buffer reservoir 176 is generally disposed between the electrolyte reservoir and the outermost ion selective membrane 180. The buffer reservoir 176 may take a variety of forms capable of temporarily retaining the buffer material 178. For example, the buffer reservoir 176 may take the form of a cavity, a porous membrane, or a gel. The buffer material 178 may supply ions for transfer through the outermost ion selective membrane 142 to the biological interface 118. Consequently, the buffer material 178 may comprise, for example, a salt (e.g., NaCl).

The outermost ion selective membrane 180 of the counter electrode assembly 114 may take a variety of forms. For example, the outermost ion selective membrane 180 may take the form of a charge selective ion exchange membrane. Typically, the outermost ion selective membrane 180 of the counter electrode assembly 114 is selective to ions with a charge or polarity opposite to that of the outermost ion selective membrane 138 of the active electrode assembly 112. The outermost ion selective membrane 180 is therefore an anion exchange membrane, which substantially passes anions and blocks cations, thereby prevents the back flux of the cations from the biological interface. Examples of suitable ion exchange membranes include the previously discussed membranes.

Alternatively, the outermost ion selective membrane 180 may take the form of a semi-permeable membrane that substantially passes and/or blocks ions based on size or molecular weight of the ion.

The outer release liner (not shown) may generally be positioned overlying or covering an outer surface 184 of the outermost ion selective membrane 180. The outer release liner may protect the outermost ion selective membrane 180 during storage, prior to application of an electromotive force or current. The outer release liner may be a selectively releasable liner made of waterproof material, such as release liners commonly associated with pressure sensitive adhesives. In some embodiments, the outer release liner may be coextensive with the outer release liner (not shown) of the active electrode assembly 112.

The transdermal delivery device 102 may further comprise an inert molding material 186 adjacent exposed sides of the various other structures forming the active and counter electrode assemblies 112, 114. The molding material 186 may advantageously provide environmental protection to the various structures of the active and counter electrode assemblies 112, 114. Enveloping the active and counter electrode assemblies 112, 114 is a housing material 190.

The active and counter electrode assemblies 112, 114 may be positioned on the biological interface (not shown). Positioning on the biological interface may close the circuit, allowing electromotive force to be applied and/or current to flow from the portable power supply system 14, to the active electrode assembly 112, to the biological interface and to the counter electrode assembly 114.

In use, the outermost active electrode ion selective membrane 138 may be placed directly in contact with the biological interface. Alternatively, an interface-coupling medium (not shown) may be employed between the outermost active electrode ion selective membrane 122 and the biological interface. The interface-coupling medium may take, for example, the form of an adhesive and/or gel. The gel may take, for example, the form of a hydrating gel or a hydrogel. If used, the interface-coupling medium should be permeable by the active agent 136, 140, 142.

In some embodiments, the portable power supply system 14 is selected to provide sufficient voltage, current, and/or duration to ensure delivery of the one or more active agents 136, 140, 142 from the reservoir 134 and across a biological interface (e.g., a membrane) to impart the desired physiological effect. The portable power supply system 14 may, for example, provide a voltage of 12.8 V DC, with tolerance of 0.8 V DC, and a current of 0.3 mA. The portable power supply system 14 may be selectively, electrically coupled to the active and counter electrode assemblies 112, 114 via a control circuit, for example, via carbon fiber ribbons. The transdermal delivery device 102 may include discrete and/or integrated circuit elements to control the voltage, current, and/or power delivered to the electrode assemblies 112, 114. For example, the transdermal delivery device 102 may include a diode to provide a constant current to the electrode elements 124, 168.

The control circuit 32 is electrically coupleable to provide a voltage across the counter and the active electrode elements 168, 124, of the transdermal delivery device 102, from the power source 30 carried by the power supply during at least a portion of a period when the power supply is magnetically-releasably coupled to the active agent delivery component 12. In some embodiments, the control circuit 32 takes the form of a programmable control circuit operable to provide at least a first active agent delivery profile. In some embodiments, the control circuit 32 takes the form of a programmable control circuit operable to provide at least one active agent delivery profile associated with a control delivery or a sustain delivery of the active agent. Further examples of programmable delivery profiles include programmable current profiles tailored to the delivery of specific active agents, ramp-up and auto-shut off functionality, bolus dose followed by a dose delivery regimen, digital pulse-width modulation of the current source tailored to drug delivery requirements (e.g., pseudo constant current using pulse width modulation), and the like.

As suggested above, the one or more active agents 136, 140, 142 may take the form of one or more ionic, cationic, ionizeable, and/or neutral drug or other therapeutic agent. Consequently, the poles or terminals of the portable power supply system 14 and the selectivity of the outermost ion selective membranes 138, 180 and inner ion selective membranes 130, 174 are selected accordingly.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small, more highly charged active agents under the influence of these forces. These higher molecular weight active agents may thus be carried through the biological interface into the underlying tissues primarily via electroosmotic solvent flow. In certain embodiments, the high molecular weight polyelectrolytic active agents may be proteins, polypeptides, or nucleic acids. In other embodiments, the active agent may be mixed with another agent to form a complex capable of being transported across the biological interface via one of the motive methods described above.

In some embodiments, the active agent delivery component 12 may further include a plurality of microneedles (not shown) in fluidic communication with the active electrode assembly 112, and positioned between the active electrode assembly 112 and the biological interface. The microneedles may be individually provided or formed as part of one or more arrays. In some embodiments, the microneedles are integrally formed from one of the substrates. The microneedles may take a solid and permeable form, a solid and semi-permeable form, and/or a solid and non-permeable form. In some other embodiments, solid, non-permeable, microneedles may further comprise grooves along their outer surfaces for aiding the transdermal delivery of one or more active agents. In some other embodiments, the microneedles may take the form of hollow microneedles. In some embodiments, the hollow microneedles may be filled with ion exchange material, ion selective materials, permeable materials, semi-permeable materials, solid materials, and the like. The microneedles may be used, for example, to deliver a variety of pharmaceutical compositions, molecules, compounds, active agents, and the like to a living body via a biological interface, such as skin or mucous membrane. In certain embodiments, pharmaceutical compositions, molecules, compounds, active agents, and the like may be delivered into or through the biological interface. For example, in delivering pharmaceutical compositions, molecules, compounds, active agents, and the like via the skin, the length of the microneedle, either individually or in arrays, and/or the depth of insertion may be used to control whether administration of pharmaceutical compositions, molecules, compounds, active agents, and the like is only into the epidermis, through the epidermis to the dermis, or subcutaneous. In certain embodiments, the microneedles may be useful for delivering high-molecular weight active agents, such as those comprising proteins, peptides and/or nucleic acids, and corresponding compositions thereof. In certain embodiments, for example, wherein the fluid is an ionic solution, the microneedles can provide electrical continuity between the portable power supply system 14 and the tips of the microneedles. In some embodiments, the microneedles, either individually or in arrays, may be used to dispense, deliver, and/or sample fluids through hollow apertures, through the solid permeable or semi permeable materials, or via external grooves. The microneedles may further be used to dispense, deliver, and/or sample pharmaceutical compositions, molecules, compounds, active agents, and the like by iontophoretic methods, as disclosed herein. The microneedles may be sized and shaped to penetrate the outer layers of skin to increase its permeability and transdermal transport of pharmaceutical compositions, molecules, compounds, active agents, and the like. In some embodiments, the microneedles are sized and shaped with an appropriate geometry and sufficient strength to insert into a biological interface (e.g., the skin or mucous membrane on a subject, and the like), and thereby increase a trans-interface (e.g., transdermal) transport of pharmaceutical compositions, molecules, compounds, active agents, and the like.

The microneedles may be manufactured from a variety of materials, including ceramics, elastomers, epoxy photoresist, glass, glass polymers, glass/polymer materials, metals (e.g., chromium, cobalt, gold, molybdenum, nickel, stainless steel, titanium, tungsten steel, and the like), molded plastics, polymers, biodegradable polymers, non-biodegradable polymers, organic polymers, inorganic polymers, silicon, silicon dioxide, polysilicon, silicon rubbers, silicon-based organic polymers, superconducting materials (e.g., superconductor wafers), and the like, as well as combinations, composites, and/or alloys thereof. Techniques for fabricating the microneedles are well known in the art and include, for example, electro-deposition, electro-deposition onto laser-drilled polymer molds, laser cutting and electro-polishing, laser micromachining, surface micro-machining, soft lithography, x-ray lithography, LIGA techniques (e.g., X-ray lithography, electroplating, and molding), injection molding, conventional silicon-based fabrication methods (e.g., inductively coupled plasma etching, wet etching, isotropic and anisotropic etching, isotropic silicon etching, anisotropic silicon etching, anisotropic GaAs etching, deep reactive ion etching, silicon isotropic etching, silicon bulk micromachining, and the like), complementary-symmetry/metal-oxide semiconductor (CMOS) technology, deep x-ray exposure techniques, and the like. Some or all of the teachings therein may be applied to microneedle devices, their manufacture, and their use in iontophoretic applications. In some techniques, the physical characteristics of the microneedles depend on, for example, the anodization conditions (e.g., current density, etching time, HF concentration, temperature, bias settings, and the like) as well as substrate properties (e.g., doping density, doping orientation, and the like).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: U.S. patent application Ser. No. 10/488970, filed Aug. 24, 2004; U.S. provisional patent application Ser. No. 60/627,952, filed Nov. 16, 2004; U.S. patent application Ser. No. 11/947667, filed Nov. 29, 2007; U.S. provisional patent application Ser. No. 60/868,317 filed Dec. 12, 2006; and U.S. provisional patent application Ser. No. 60/949,810 filed Jul. 13, 2007.

As one of skill in the art would readily appreciate, the present disclosure comprises methods of treating a subject by any of the compositions and/or methods described herein.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs, or other structures. Still other embodiments may employ additional ones of the membranes, reservoirs, and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

What is claimed is:

1. A medical device, comprising:
a first portion including at least one electrode selectively operable to provide an electrical potential and including at least a first element of a magnetic coupler;
a second portion including at least one battery cell and including at least a second element of the magnetic coupler, the second element of the magnetic coupler complementary to the first element of the magnetic coupler, the second portion of the medical device selectively removably magnetically coupleable to the first portion of the medical device via a magnetic interaction between the first and the second elements of the magnetic coupler; and
a ferrous element physically carried by one of the first or the second portions, wherein at least one of the first or the second elements is a magnet and the ferrous element is physically positioned between the battery and the magnet to steer a magnetic flux of the magnet away from the battery.

2. The medical device of claim 1 wherein the second element of the magnetic coupler is the magnet and the first element of the magnetic coupler is a ferrous metal.

3. The medical device of claim 1 wherein the second element of the magnetic coupler is the magnet and the first element of the magnetic coupler is another magnet, where opposite magnet poles of the two magnets face one another when the second portion of the medical device is magnetically coupled to the first portion of the medical device.

4. The medical device of claim 1 wherein the first element of the magnetic coupler is the magnet and the second element of the magnetic coupler is one of a ferrous metal or another magnet.

5. The medical device of claim 1 wherein the second portion further includes a control circuit configured to control the electrical potential provided at the at least one electrode of the first portion.

6. The medical device of claim 1 wherein the first portion further includes an active agent reservoir positioned such that the electrical potential provided at the at least one electrode of the first portion drives an active agent from the active agent reservoir.

7. The medical device of claim 1 wherein the battery is disc shaped and the ferrous element is disc shaped.

8. The medical device of claim 7 wherein a diameter of the ferrous element is at least approximately equal to a diameter of the battery.

9. The medical device of claim 8 wherein the magnet is disc shaped and the diameter of the ferrous element is greater than a diameter of the magnet.

10. The medical device of claim 1 wherein at least one of the first or the second portions of the medical device includes a non-ferrous spacer, the non-ferrous spacer positioned between the ferrous element and the battery.

11. The medical device of claim 1 wherein the magnet takes the form of at least one permanent magnet.

12. The medical device of claim 11 wherein the at least one permanent magnet is at least one of a high-energy flexible magnet, a neodymium magnet, a ceramic magnet, a samarium cobalt magnet, or an alnico magnet.

13. The medical device of claim 1 wherein the first portion further includes a first and a second electrical contact, and wherein the second portion includes a complementary first and a complementary second electrical contact, the first and the second electrical contacts positioned with respect to the first element of the magnetic coupler and the complementary first and the complementary second electrical contacts positioned with respect to the second element of the magnetic coupler such that the first complementary electrical contact is in electrically conductive communication with the first electrical contact and the second complementary electrical contact is in electrically conductive communication with the second electrical contact when the second portion of the medical device is magnetically coupled to the first portion of the medical device.

14. An apparatus, comprising:
a battery holder configured to hold a battery;
a first coupling magnet physically coupled to the battery holder and positioned proximate the battery holder; and
a ferrous element positioned between the battery holder and the first coupling magnet to steer a magnetic flux of the first coupling magnet away from the battery holder.

15. The apparatus of claim 14, further comprising:
a non-ferrous spacer positioned between the ferrous element and the battery holder.

16. The apparatus of claim 14, further comprising:
a control circuit coupled to control at least one of a current or a voltage delivered from the battery;

17. The apparatus of claim 14, further comprising:
the battery, received in the battery holder.

18. The apparatus of claim 14 wherein the apparatus is a removable power source magnetically coupleable to another device via the first coupling magnet.

19. The apparatus of claim 14 wherein the apparatus is a medical device, and further comprises:
at least one electrode operable to provide an electrical potential when coupled to the battery.

20. The apparatus of claim 19 wherein the medical device is a transdermal active agent delivery device, and further comprising:
at least one active agent reservoir that stores an active agent.

21. The apparatus of claim 19 wherein the at least one active agent reservoir that stores an active agent is carried by a first portion that is removable attachable to a second portion which carries the battery holder.

22. The apparatus of claim 14, further comprising:
at least two electrical contacts accessible from an exterior of the apparatus, the electrical contacts in electrical communication with the battery when the battery is held by the battery holder.
